(19) United States
(12) Patent Application Publication
(10) Pub. No.: US 2006/0153819 A1
(43) Pub. Date: Jul. 13, 2006

(54) Bone marrow stromal cells for immunoprotection of transplanted neural stem cells

(75) Inventor: Kevin R. McIntosh, Ellicott City, MD (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Cognate Therapeutics, Inc.

(21) Appl. No.: 11/328,976

(22) Filed: Jan. 10, 2006

Related U.S. Application Data
(60) Provisional application No. 60/643,436, filed on Jan. 11, 2005.

(51) Int. Cl. A61K 35/30; A61K 35/14; C12N 5/08

(52) U.S. Cl. 424/93.7; 435/368; 435/372

(57) ABSTRACT

The present invention encompasses methods and compositions for reducing an immune response to a transplant recipient receiving NSCs by treating said recipient with an amount of bone marrow stromal effective to reduce or inhibit host rejection of the NSCs.

### CROSS-REFERENCE TO RELATED APPLICATIONS

The present application is entitled to priority under 35 U.S.C. § 19(e), to U.S. Provisional Application No. 60/643,436, filed on Jan. 11, 2005, which application is hereby incorporated herein by reference in its entirety.

### BACKGROUND OF THE INVENTION

Mammalian neurological damage having as its genesis trauma, tumor formation or a genetic or other component, is very difficult to treat and/or reverse. One treatment for neurological damage to the central nervous system is neurotransplantation of a variety of cells. Neurotransplantation has been used to explore the development, plasticity, and regeneration of the central nervous system (McKay, 1997, Science 276:66-71). Also, neurotransplantation has been used to effect the repair and functional restoration of diseased and damaged nervous tissues (Bjorklund, 1993, Nature 362:414-415; Olson, 1997, Nature Med. 3:1329-1335; Spencer et al., 1992, N. Engl. J. Med. 327:1541-1548: Freed et al., 1992, N. Engl. J. Med 327:1549-1555; Kordower et al., 1995, N. Engl. J. Med. 332:1118-1124; Defer et al., 1996, Brain 119:41-50; Lopez-Lozano et al., 1997, Transp. Proc. 29:977-980; Rosenstein, 1995, Exp. Neurol. 33:106; Turner et al., 1993, Neurosurg. 33:1031-1037; Kang et al., 1993, J. Neurosci. 13:5203-5211; Andersson et al., 1993, Int. J. Dev. Neurosci. 11:555-568; Sanberg et al., 1997, Nature Med. 3:1129-1132). For example, a series of human patients with Parkinson's disease have been treated by neurotransplantation of mesencephalic cells obtained from 6 to 9 week old abortuses of human fetuses (Spencer et al., 1992, N. Engl. J. Med. 327:1541-1548: Freed et al., 1992, N. Engl. J. Med 327:1549-1555; Kordower et al., 1995, N. Engl. J. Med. 332:1118-1124; Defer et al., 1996, Brain 119:41-50; Lopez-Lozano et al., 1997, Transp. Proc. 29:977-980). It was observed that some of the patients exhibited significant improvement both in clinical symptoms and in the synthesis of dopamine (Spencer et al., 1992, N. Engl. J. Med. 327:1541-1548; Freed et al., 1992, N. Engl. J. Med 327:1549-1555; Kordower et al., 1995, N. Engl. J. Med. 332:1118-1124; Defer et al., 1996, Brain 119:41-50). However, the process of obtaining fetal tissue for therapeutic uses has presented major logistic and ethical barriers (Rosenstein, 1995, Exp. Neurol. 33:106; Turner et al., 1993, Neurosurg. 33:1031-1037). Also, only about 5% to 10% of dopaminergic neurons survive, because of adverse immune reaction to the same (Lopez-Lozano et al., 1997, Transp. Proc. 29:977-980) and because the fetal tissue is primarily dependent on lipid instead of glycolytic metabolism (Rosenstein, 1995, Exp. Neurol. 33:106). For these reasons, attempts have been made to develop alternative cells such as fibroblasts (Kang et al., 1993, J. Neurosci. 13:5203-5211), fetal astrocytes (Andersson et al., 1993, Int. J. Dev. Neurosci. 11:555-568), and sertoli cells (Sanberg et al., 1997, Nature Med. 3:1129-1132) for neurotransplantation.

It is believed that in order to treat diseases, disorders, or conditions of the central nervous system, such as for example brain tumors, brain trauma, Huntington's disease, Alzheimer's disease, Parkinson's disease, and spinal cord injury, by transplantation, donor cells should be easily available, capable of rapid expansion in culture, immunologically inert, capable of long term survival and integration in the host brain tissue, and amenable to stable transfection and long-term expression of exogenous genes (Bjorklund, 1993, Nature 362:414-415; Olson, 1997, Nature Med. 3:1329-1335). Donor cells meeting these criteria are not currently available.

During development of the central nervous system (“CNS”), multipotent precursor cells, also known as neural stem cells (NSCs), proliferate, giving rise to transiently dividing progenitor cells that eventually differentiate into the cell types that compose the adult brain. Stem cells (from other tissues) have classically been defined as having the ability to self-renew (i.e., form more stem cells), to proliferate, and to differentiate into multiple different phenotypic lineages. In the case of neural stem cells, the different phenotypic lineages include neurons, astrocytes and oligodendrocytes.

NSCs have been isolated from several mammalian species, including mice, rats, pigs and humans (WO 93/01275, WO 94/09119, WO 94/10292, WO 94/16718; Cattaneo et al., 1996, Mol. Brain Res. 42:161-66. Human CNS neural stem cells, like their rodent homologs, when maintained in a mitogen-containing (typically epidermal growth factor or epidermal growth factor plus basic fibroblast growth factor) and serum-free culture medium, grow in suspension culture to form aggregates of cells known as “neurospheres”. It has been observed that human neural stem cells have doubling rates of about 30 days (Cattaneo et al., 1996, Mol Brain Res. 42:161-66). Upon removal of the mitogen(s), the stem cells can differentiate into neurons, astrocytes and oligodendrocytes.

The mammalian immune system plays a central role in protecting individuals from infectious agents and preventing tumor growth. However, the same immune system can produce undesirable effects such as the rejection of cell, tissue and organ transplants from unrelated donors. The immune system does not distinguish beneficial intruders, such as a transplanted tissue, from those that are harmful, and thus the immune system rejects transplanted tissues or organs. Rejection of transplanted organs is generally mediated by alloreactive T cells present in the host which recognize donor alloantigens or xenoantigens.

The transplantation of cells, tissues, and organs between genetically disparate individuals invariably results in the risk of graft rejection. Nearly all cells express products of the major histocompatibility complex, MHC class I molecules. Further, many cell types can be induced to express MHC class II molecules when exposed to inflammatory cytokines. Additional immunogenic molecules include those derived from minor histocompatibility antigens such as Y chromosome antigens recognized by female recipients. Rejection of allografts is mediated primarily by T cells of both the CD4 and CD8 subclasses (Rosenberg et al., 1992, Annu. Rev. Immunol. 10:333). Alloreactive CD4+ T cells produce cytokines that exacerbate the cytolytic CD8 response to alloantigen. Within these subclasses, competing subpopulations of cells develop after antigen stimulation that are characterized by the cytokines they produce. Th1 cells, which produce IL-2 and IFN-γ, are primarily involved in allograft rejection (Mossmann et al., 1989, Annu. Rev. Immunol. 7:145). Th2 cells, which produce IL-4 and IL-10, can down-regulate Th1 responses through IL-10 (Fiorentino et., 1989, J. Exp. Med. 170:2081). Indeed, much effort has been expended to divert undesirable Th1 responses toward the Th2 pathway. Undesirable alloreactive T cell responses in patients (allograft rejection, graft-versus-host disease) are typically handled with immunosuppressive drugs such as prednisone, azathioprine, and cyclosporine A. Unfortunately, these drugs generally need to be maintained for the life of the patient and they have a multitude of dangerous side effects including generalized immunosuppression. A much better approach than pan immunosuppression is to induce specific or localized suppression to donor cell alloantigens, leaving the remaining immune system intact.

It is believed that there are numerous ways to induce immunologic tolerance to alloantigens that would allow transplantation of allogeneic stem cells. Unfortunately, many of the approaches that have worked well in rodent animal models have not been successful when applied to nonhuman primates or humans. Similarly, the use of nuclear transfer to create clones of embryonic stem cells genetically identical to the recipient has been problematic for higher species, although limited success was recently reported for humans (Hwang et al., 2004, Science 303:1669). It is not clear how this technology could be applied to engineering other types of stem cells, and whether the time required for manipulation and expansion would obviate their usefulness.

Stem cells were reported to exhibit a low degree of immunogenicity, possibly due to their immature state of differentiation and immunoregulatory properties. Rat embryonic stem cell-like lines express low levels of MHC class I antigens and they are negative for expression of MHC class II molecules and CD80(B7-1)/86(B7-2) costimulatory molecules (Fandrich et al., 2002, Nat. Med. 8:171). These cells engrafted in the liver of immunocompetent allogeneic recipient rats when injected into the portal vein. Engraftment was attributed to lack of costimulatory molecules and the expression of FasL by the stem cell lines. Activated T cells express the Fas receptor, thus rendering them susceptible to apoptosis by the stem cell lines. Whether these properties are shared by other embryonic stem cell lines is currently unknown as transplanted fetal and embryonic stem cell-derived tissues are frequently rejected by the recipient's immune system (Bradley et al., 2002, Nat. Rev. 2:859, Kaufman et al., 2000, E-biomed 1:11). Neural stem cells derived from rodents express low or negligible levels of MHC class I or class II antigens (McLaren et al., 2001, J. Neuroimmunol 112:35), but these cells are usually rejected after implantation into allogeneic recipients unless immunosuppressive drugs are used (Mason et al., 1986, Neuroscience 19:685, Sloan et al., 1991, Trends Neurosci. 14:341, Wood et al., 1996, Neuroscience 70:775). Rejection may be initiated after MHC molecules are up-regulated on cell membranes after exposure to inflammatory cytokines of the IFN family (McLaren et al., 2001, J. Neuroimmunol 112:35).

A major goal in organ transplantation is the permanent engraftment of the donor organ without inducing a graft rejection immune response generated by the recipient, while preserving the immunocompetence of the recipient against other foreign antigens. Typically, in order to prevent host rejection responses, nonspecific immunosuppressive agents such as cyclosporine, methotrexate, steroids and FK506 are used. These agents must be administered on a daily basis and if administration is stopped, graft rejection usually results. However, a major problem in using nonspecific immunosuppressive agents is that they function by suppressing all aspects of the immune response, thereby greatly increasing a recipient's susceptibility to infection and other diseases, including cancer. Furthermore, despite the use of immunosuppressive agents, graft rejection still remains a major source of morbidity and mortality in human organ transplantation. Most human transplants fail within 10 years without permanent graft acceptance. Only 50% of heart transplants survive 5 years and 20% of kidney transplants survive 10 years. (Opelz et al., 1981, Lancet 1:1223).

It is currently believed that a successful transplantation is dependent on the prevention and/or reduction of an unwanted immune response by the host to a transplant mediated by immune effector cells to avert host rejection of donor tissue. Also advantageous for a successful transplantation is a method to eliminate or reduce an unwanted immune response by the donor tissue against a recipient tissue known as graft-versus-host disease. Thus, there is long-felt need for methods to suppress or otherwise prevent an unwanted immune response associated with transplantation of cells, tissues, and organs between genetically disparate individuals. The present invention meets this need.

### BRIEF SUMMARY OF THE INVENTION

The invention includes compositions and methods for transplantation of neural stem cells (NSCs). The invention also includes compositions and methods for treating a patient receiving transplantation of NSCs.

The invention includes a method of treating a transplant recipient, wherein said transplant is a neural stem cell (NSC), to reduce in said recipient an immune response of effector cells against an alloantigen to said effector cells.

In one aspect, the method comprises administering to a transplant recipient, bone marrow stromal cells (BMSCs) in an amount effective to reduce an immune response of effector cells against an alloantigen to said effector cells, whereby in the transplant recipient said effector cells have a reduced immune response against said alloantigen.

In another aspect, the effector cells are T cells.

In a further aspect, the T cells are from a donor and the alloantigen is from a recipient.

In yet a further aspect, the T cells are from a recipient and the alloantigen is from a donor.

In another aspect, the T cells are present in the transplant.

In a further aspect, BMSCs are expanded in culture prior to administering the BMSCs to a transplant recipient.

In one aspect, the effector cells are T cells that have been activated prior to administration of BMSCs, and further wherein the immune response is the reactivation of the T cells from the donor.

In yet another aspect, BMSCs are administered to the transplant recipient to treat rejection of the transplant by the recipient.

In a further aspect, BMSCs are derived from a human.

In another aspect, BMSCs are derived from a mouse or a rat.

In yet another aspect, NSCs are derived from a human.

In a further aspect, an immunosuppressive agent is administered to the transplant recipient.

In one aspect, BMSCs are administered to the recipient prior to the administration of a transplant to the recipient.

In another aspect, BMSCs are administered to the recipient concurrently with the transplant.

In a further aspect, BMSCs are administered as part of the transplant.

In yet another aspect, BMSCs are administered to the recipient subsequent to the transplantation of the transplant.

In one aspect, BMSCs are administered intravenously to the recipient.

In another aspect, the effector cells are cells of a recipient of the transplant.

In a further aspect, BMSCs are genetically modified.

The invention includes a method for treating a transplant recipient, wherein said transplant is a neural stem cell (NSC), to reduce in said recipient an immune response of effector cells against an alloantigen to the effector cells, comprising transplanting to a transplant recipient, NSCs with BMSCs in an amount effective to reduce an immune response of effector cells against an alloantigen to the effector cells, whereby in the transplant recipient the effector cells have a reduced immune response against the alloantigen.

In one aspect, the effector cells are T cells.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiment(s) which are presently preferred. It should be understood, however, that the invention is not limited to the precise arrangements and instrumentalities shown.

FIG. 1 is a graph depicting the immunogenicity of neural stem cells (NSCs). FIG. 1 demonstrates that NSCs stimulated proliferation of allogeneic T cells.

FIG. 2 is a graph depicting the immunogenicity of bone marrow stromal cells (BMSCs) as evaluated by one-way mixed lymphocyte reaction (MLR). FIG. 2 demonstrates that BMSCs do not stimulate allogeneic T cell proliferation.

FIG. 3 is a graph demonstrating that BMSCs do not induce expression of the activated molecule CD 25 on allogeneic T cells.

FIG. 4 is a graph depicting suppression of the MLR by BMSCs.

### DETAILED DESCRIPTION

The present invention relates to the discovery that bone marrow stromal cells (BMSCs) possess novel immunological characteristics and therefore can be useful in transplantation of a transplant, for example a biocompatible lattice or a donor tissue, organ or cell, by reducing and/or eliminating an immune response against the transplant by the recipient's own immune system. As described more fully below, BMSCs play a role in inhibiting and/or preventing allograft rejection of a transplant.

In addition, the data disclosed herein demonstrate that BMSCs are useful for the inhibition and/or prevention of an unwanted immune response by a donor transplant, for example, a biocompatible lattice or a donor tissue, organ or cell, against a recipient tissue known as graft-versus-host disease.

Accordingly, the present invention encompasses methods and compositions for reducing and/or eliminating an immune response to a transplant in a recipient by treating the recipient with an amount of BMSCs effective to reduce or inhibit host rejection of the transplant. Also encompassed are methods and compositions for reducing and/or eliminating an immune response in a host by the foreign transplant against the host, i.e., graft versus host disease, by treating the donor transplant and/or recipient of the transplant bone marrow stromal cells in order to inhibit or reduce an adverse response by the donor transplant against the recipient.

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

As used herein, the term “autologous” is meant to refer to any material derived from the same individual to which it is later to be re-introduced into the individual.

As used herein, the term “biocompatible lattice,” is meant to refer to a substrate that can facilitate formation into three-dimensional structures conducive for tissue development. Thus, for example, cells can be cultured or seeded onto such a biocompatible lattice, such as one that includes extracellular matrix material, synthetic polymers, cytokines, growth factors, etc. The lattice can be molded into desired shapes for facilitating the development of tissue types. Also, at least at an early stage during culturing of the cells, the medium and/or substrate is supplemented with factors (e.g., growth factors, cytokines, extracellular matrix material, etc.) that facilitate the development of appropriate tissue types and structures.

As used herein, the term “bone marrow stromal cells,” “stromal cells,” “mesenchymal stem cells” or “MSCs” are used interchangeably and refer to the small fraction of cells in bone marrow which can serve as stem cell-like precursors to osteocytes, chondrocytes, and adipocytes. Bone marrow stromal cells have been studied extensively (Castro-Malaspina et al., 1980, Blood 56:289-30125; Piersma et al., 1985, Exp. Hematol 13:237-243; Simmons et al., 1991, Blood 78:55-62; Beresford et al., 1992, J. Cell. Sci. 102:341-351; Liesveld et al., 1989, Blood 73:1794-1800; Liesveld et al., Exp. Hematol 19:63-70; Bennett et al., 1991, J. Cell. Sci. 99:131-139). Bone marrow stromal cells may be derived from any animal. In some embodiments, stromal cells are derived from primates, preferably humans. In addition, BMSCs are able to suppress alloreactive T cell proliferation during an immune response. For example, BMSCs can suppress a mixed lymphocyte reaction (MLR) between allogeneic T cells and peripheral blood mononuclear cells (PBMCs).

“Neural stem cell” or “NSC” is used herein to refer to undifferentiated, multipotent, self-renewing neural cell. A neural stem cell is a clonogenic multipotent stem cell which is able to divide and, under appropriate conditions, has self-renewal capability and can terminally differentiate into neurons, astrocytes, and oligodendrocytes. Hence, the neural stem cell is “multipotent” because stem cell progeny have multiple differentiation pathways. A neural stem cell is capable of self maintenance, meaning that with each cell division, one daughter cell will also be, on average, a stem cell.

“Graft” refers to a cell, tissue, organ or otherwise any biological compatible lattice for transplantation.

“Allogeneic” refers to a graft derived from a different animal of the same species.

“Xenogeneic” refers to a graft derived from an animal of a different species.

“Transplant” refers to a biocompatible lattice or a donor tissue, organ or cell, to be transplanted. An example of a transplant may include but is not limited to skin cells or tissue, bone marrow, and solid organs such as heart, pancreas, kidney, lung and liver. Preferably, the transplant is a human neural stem cell.

As defined herein, an “allogeneic bone marrow stromal cell (BMSC)” is obtained from a different individual of the same species as the recipient.

“Donor antigen” refers to an antigen expressed by the donor tissue to be transplanted into the recipient.

“Alloantigen” is an antigen that differs from an antigen expressed by the recipient.

As used herein, an “effector cell” refers to a cell which mediates an immune response against an antigen. In the situation where a transplant is introduced into a recipient, the effector cells can be the recipient's own cells that elicit an immune response against an antigen present in the donor transplant. In another situation, the effector cell can be part of the transplant, whereby the introduction of the transplant into a recipient results in the effector cells present in the transplant eliciting an immune response against the recipient of the transplant.

By the term “treating a transplant recipient to reduce in said recipient an immune response of effector cells against an alloantigen to the effector cells,” as the phrase is used herein, is meant decreasing the endogenous immune response against the alloantigen in a recipient by any method, for example administering BMSCs to a recipient, compared with the endogenous immune response in an otherwise identical animal which was not treated with BMSCs. The decrease in endogenous immune response can be assessed using the methods disclosed herein or any other method for assessing endogenous immune response in an animal.

As used herein, a “therapeutically effective amount” is the amount of BMSCs which is sufficient to provide a beneficial effect to the subject to which the BMSCs are administered.

As used herein “endogenous” refers to any material from or produced inside an organism, cell or system.

“Exogenous” refers to any material introduced from or produced outside an organism, cell, or system.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like. “Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

Description

The present invention relates to the discovery that when bone marrow stromal cells (BMSCs) are contacted with T cells obtained from a different individual (allogeneic T cells), the allogeneic T cells do not proliferate. Prior art dogma suggests that when T cells are mixed with any other cell, T cell proliferation ensues. The mixed lymphocyte reaction (MLR) is a standard assay used to evaluate immunogenicity. The data disclosed herein demonstrate that T cells derived from an individual are not responsive to BMSCs obtained from a different individual. Therefore, based upon the disclosure herein, BMSCs are not immunogenic to the immune system with respect to manifesting a T cell response.

In addition to the non-immunogenic phenotype of BMSCs with respect to T lymphocytes in a different individual, the present invention also relates to the discovery that BMSCs can suppress an MLR between allogeneic cells, for example between T cells from one individual and peripheral blood mononuclear cells (PBMCS) from another individual. These unexpected results demonstrate that BMSCs can actively reduce the allogeneic T cell response in MLRs between T cells and PBMCs from different individuals. Moreover, as discussed in more detail elsewhere herein, this reduction is observed to occur in a dose dependent manner. This shows that BMSCs can be used as a therapy to inhibit host rejection of a transplant, and in addition, prevent or otherwise inhibit graft versus host disease following transplantation.

I. Therapy to Inhibit Host Rejection of a Transplant

The present invention includes a method of using BMSCs as a therapy to inhibit host rejection of a transplant. The invention is based on the discovery that BMSCs do not stimulate allogeneic T cell proliferation. In addition, BMSCs were observed to suppress T cell proliferation in an MLR reaction.

One skilled in the art would appreciate, based upon the disclosure provided herein, that the ability of BMSCs to suppress an allogeneic T cell response is not limited to an MLR between T cells and PBMCs from disparate individuals. Rather, the BMSCs can be exploited to include suppression of an MLR between T cells and any type of cell from a different individual. For example, the MLR between T cells and a neural stem cell (NSC), a liver cell, a cardiac cell, a chondrocyte, a kidney cell, an adipose cell and the like can be suppressed using BMSCs. Preferably, BMSCs are used to inhibit host rejection of transplanted human NSCs.

The present invention encompasses a method of reducing and/or eliminating an immune response to a transplant in a recipient by administering to the recipient of the transplant an amount of BMSCs effective to reduce or inhibit host rejection of the transplant. Without wishing to be bound to any particular theory, the BMSCs that are administered to the recipient of the transplant inhibit the activation and proliferation of the recipient's T cells.

The transplant includes a biocompatible lattice or a donor tissue, organ or cell, to be transplanted. An example of a transplant may include but is not limited to skin cells or tissue, bone marrow, and solid organs such as heart, pancreas, kidney, lung and liver. Preferably, the transplant is a human neural stem cell.

Based upon the disclosure provided herein, BMSCs can be obtained from any source, for example, from the tissue donor, the transplant recipient or an otherwise unrelated source (a different individual or species altogether). The BMSCs may be autologous with respect to the T cells (obtained from the same host) or allogeneic with respect to the T cells. In the case where the BMSCs are allogeneic, the BMSCs may be autologous with respect to the transplant to which the T cells are responding to, or the BMSCs may be obtained from an individual that is allogeneic with respect to both the source of the T cells and the source of the transplant to which the T cells are responding to. In addition, the BMSCs may be xenogeneic to the T cells (obtained from an animal of a different species), for example rat BMSCs may be used to suppress activation and proliferation of human T cells in MLRs.

In a further embodiment, BMSCs used in the present invention can be isolated, from the bone marrow of any species of mammal, including but not limited to, human, mouse, rat, ape, gibbon, bovine. Preferably, the BMSCs are isolated from a human, a mouse, or a rat. More preferably, the BMSCs are isolated from a human.

Based upon the present disclosure, BMSCs can be isolated and expanded in culture in vitro to obtain sufficient numbers of cells for use in the methods described herein. For example, BMSCs can be isolated from human bone marrow and cultured in complete medium (DMEM low glucose containing 4 mM L-glutamine, 10% FBS, and 1% penicillin/streptomycin). However, the invention should in no way be construed to be limited to any one method of isolating and culturing BMSCs. Rather, any method of isolating and culturing BMSCs should be construed to be included in the present invention.

Any medium capable of supporting BMSCs in vitro may be used to culture the BMSCs. Media formulations that can support the growth of BMSCs include, but are not limited to, Dulbecco's Modified Eagle's Medium (DMEM), alpha modified Minimal Essential Medium (αMEM), and Roswell Park Memorial Institute Media 1640 (RPMI Media 1640) and the like. Typically, 0 to 20% fetal bovine serum (FBS) or 1-20% horse serum is added to the above medium in order to support the growth of BMSCs. However, a defined medium can also be used if the growth factors, cytokines, and hormones necessary for culturing BMSCs are provided at appropriate concentrations in the medium. Media useful in the methods of the invention may contain one or more compounds of interest, including but not limited to antibiotics, mitogenic or differentiation compounds useful for the culturing of BMSCs. The cells may be grown at temperatures between 27° C. to 40° C., preferably 31° C. to 37° C., and more preferably in a humidified incubator. The carbon dioxide content may be maintained between 2% to 10% and the oxygen content may be maintained between 1% and 22%. However, the invention should in no way be construed to be limited to any one method of isolating and culturing BMSCs. Rather, any method of isolating and culturing BMSCs should be construed to be included in the present invention.

Antibiotics which can be added into the medium include, but are not limited to, penicillin and streptomycin. The concentration of penicillin in the culture medium is about 10 to about 200 units per ml. The concentration of streptomycin in the culture medium is about 10 to about 200 μg/ml.

Another embodiment of the present invention encompasses the route of administering BMSCs to the recipient of the transplant. BMSCs can be administered by a route which is suitable for the placement of the transplant, i.e. a biocompatible lattice or a donor tissue, organ or cell, to be transplanted. BMSCs can be administered systemically, i.e., parenterally, by intravenous injection or can be targeted to a particular tissue or organ, such as bone marrow. BMSCs can be administered via a subcutaneous implantation of cells or by injection of the cells into connective tissue, for example, muscle.

BMSCs can be suspended in an appropriate diluent, at a concentration of from about 0.01 to about 5×10⁶ cells/ml. Suitable excipients for injection solutions are those that are biologically and physiologically compatible with the BMSCs and with the recipient, such as buffered saline solution or other suitable excipients. The composition for administration can be formulated, produced and stored according to standard methods complying with proper sterility and stability.

The dosage of the BMSCs varies within wide limits and may be adjusted to the individual requirements in each particular case. The number of cells used depends on the weight and condition of the recipient, the number and/or frequency of administrations, and other variables known to those of skill in the art.

Between about 10⁵ and about 10¹³ BMSCs per 100 kg body weight can be administered to the individual. In some embodiments, between about 1.5×10⁶ and about 1.5×10¹² cells are administered per 100 kg body weight. In some embodiments, between about 1×10⁹ and about 5×10¹¹ cells are administered per 100 kg body weight. In some embodiments, between about 4×10⁹ and about 2×10¹¹ cells are administered per 100 kg body weight. In some embodiments, between about 5×10⁸ cells and about 1×10¹ cells are administered per 100 kg body weight.

In another embodiment of the present invention, BMSCs are administered to the recipient prior to, or contemporaneously with a transplant to reduce and/or eliminate host rejection of the transplant. While not wishing to be bound to any particular theory, BMSCs can be used to condition a recipient's immune system to the transplant by administering BMSCs to the recipient, prior to, or at the same time as transplantation of the transplant, in an amount effective to reduce, inhibit or eliminate an immune response against the transplant by the recipient's T cells. The BMSCs affect the T cells of the recipient such that the T cell response is reduced, inhibited or eliminated when presented with the transplant. Thus, host rejection of the transplant may be avoided, or the severity thereof reduced, by administering BMSCs to the recipient, prior to, or at the same time as transplantation.

In yet another embodiment, BMSCs can be administered to the recipient of the transplant after the administration of the transplant. Further, the present invention comprises a method of treating a patient who is undergoing an adverse immune response to a transplant by administering BMSCs to the patient in an amount effective to reduce, inhibit or eliminate the immune response to the transplant, also known as host rejection of the transplant.

II. Therapy to Inhibit Graft Versus Host Disease Following Transplantation

The present invention includes a method of using BMSCs as a therapy to inhibit graft versus host disease following transplantation. The invention is based on the discovery that BMSCs do not stimulate allogeneic T cell proliferation. In addition, BMSCs were observed to suppress T cell proliferation in an MLR reaction.

The present invention also provides a method of reducing and/or eliminating an immune response by a donor transplant against a recipient thereof (i.e. graft versus host reaction). Accordingly, the present invention encompasses a method of contacting a donor transplant, for example a biocompatible lattice or a donor tissue, organ or cell, preferably a neural stem cell, with BMSCs prior to transplantation of the transplant into a recipient. The BMSCs serve to ameliorate, inhibit or reduce an adverse response by the donor transplant against the recipient.

As discussed elsewhere herein, BMSCs can be obtained from any source, for example, from the tissue donor, the transplant recipient or an otherwise unrelated source (a different individual or species altogether) for the use of eliminating or reducing an unwanted immune response by a transplant against a recipient of the transplant. Accordingly, BMSCs can be autologous, allogeneic or xenogeneic to the tissue donor, the transplant recipient or an otherwise unrelated source.

In an embodiment of the present invention, the transplant is exposed to BMSCs prior to transplantation of the transplant into the recipient. In this situation, an immune response against the transplant caused by any alloreactive recipient cells would be suppressed by the BMSCs present in the transplant. The BMSCs are allogeneic to the recipient and may be derived from the donor or from a source other than the donor or recipient. In some cases, BMSCs autologous to the recipient may be used to suppress an immune response against the transplant. In another case, the BMSCs may be xenogeneic to the recipient, for example mouse or rat BMSCs can be used to suppress an immune response in a human. However, it is preferable to use human BMSCs in the present invention.

In another embodiment of the present invention, the donor transplant can be “preconditioned” or “pretreated” by treating the transplant prior to transplantation into the recipient in order to reduce the immunogenicity of the transplant against the recipient, thereby reducing and/or preventing graft versus host disease. The transplant can be contacted with cells or a tissue from the recipient prior to transplantation in order to activate T cells that may be associated with the transplant. Following the treatment of the transplant with cells or a tissue from the recipient, the cells or tissue may be removed from the transplant. The treated transplant is then further contacted with BMSCs in order to reduce, inhibit or eliminate the activity of the T cells that were activated by the treatment of the cells or tissue from the recipient. Following this treatment of the transplant with BMSCs, the BMSCs may be removed from the transplant prior to transplantation into the recipient. However, some BMSCs may adhere to the transplant, and therefore, may be introduced to the recipient with the transplant. In this situation, the BMSCs introduced into the recipient can suppress an immune response against the recipient caused by any cell associated with the transplant. Without wishing to be bound to any particular theory, the treatment of the transplant with BMSCs prior to transplantation of the transplant into the recipient serves to reduce, inhibit or eliminate the activity of the activated T cells, thereby preventing restimulation, or inducing hyporesponsiveness of the T cells to subsequent antigenic stimulation from a tissue and/or cells from the recipient. One skilled in the art would understand based upon the present disclosure, that preconditioning or pretreatment of the transplant prior to transplantation may reduce or eliminate the graft versus host response.

For example, in the context of bone marrow or peripheral blood stem cell (hematopoietic stem cell) transplantation, attack of the host by the graft can be reduced, inhibited or eliminated by preconditioning the donor marrow by using the pretreatment methods disclosed herein in order to reduce the immunogenicity of the graft against the recipient. As described elsewhere herein, a donor marrow can be pretreated with BMSCs from any source, preferably with recipient BMSCs in vitro prior to the transplantation of the donor marrow into the recipient. In a preferred embodiment, the donor marrow is first exposed to recipient tissue or cells and then treated with BMSCs. Although not wishing to be bound to any particular theory, it is believed that the initial contact of the donor marrow with recipient tissue or cells function to activate the T cells in the donor marrow. Treatment of the donor marrow with the BMSCs induces hyporesponsiveness or prevents restimulation of T cells to subsequent antigenic stimulation, thereby reducing, inhibiting or eliminating an adverse affect induced by the donor marrow on the recipient.

In an embodiment of the present invention, a transplant recipient suffering from graft versus host disease may be treated by administering BMSCs to the recipient to reduce, inhibit or eliminate the severity thereof from the graft versus host disease where the BMSCs are administered in an amount effective to reduce or eliminate graft versus host disease.

In this embodiment of the invention, preferably, the recipient's BMSCs may be obtained from the recipient prior to the transplantation and may be stored and/or expanded in culture to provide a reserve of BMSCs in sufficient amounts for treating an ongoing graft versus host reaction. However, as discussed elsewhere herein, BMSCs can be obtained from any source, for example, from the tissue donor, the transplant recipient or an otherwise unrelated source (a different individual or species altogether).

III. Advantages of using BMSCs

Based upon the disclosure herein, it is envisioned that the BMSCs of the present invention can be used in conjunction with current modes, for example the use of immunosuppressive drug therapy, for the treatment of host rejection to the donor tissue or graft versus host disease. An advantage of using BMSCs in conjunction with immunosuppressive drugs in transplantation is that by using the methods of the present invention to ameliorate the severity of the immune response in a transplant recipient, the amount of immunosuppressive drug therapy used and/or the frequency of administration of immunosuppressive drug therapy can be reduced. A benefit of reducing the use of immunosuppressive drug therapy is the alleviation of general immune suppression and unwanted side effects associated with immunosuppressive drug therapy.

It is also contemplated that the cells of the present invention may be administered into a recipient as a “one-time” therapy for the treatment of host rejection of donor tissue or graft versus host disease. A one-time administration of BMSCs into the recipient of the transplant eliminates the need for chronic immunosuppressive drug therapy. However, if desired, multiple administrations of BMSCs may also be employed.

The invention described herein also encompasses a method of preventing or treating transplant rejection and/or graft versus host disease by administering BMSCs in a prophylactic or therapeutically effective amount for the prevention, treatment or amelioration of host rejection of the transplant and/or graft versus host disease. Based upon the present disclosure, a therapeutic effective amount of BMSCs is an amount that inhibits or decreases the number of activated T cells, when compared with the number of activated T cells in the absence of the administration of BMSCs. In the situation of host rejection of the transplant, an effective amount of BMSCs is an amount that inhibits or decreases the number of activated T cells in the recipient of the transplant when compared with the number of activated T cells in the recipient prior to administration of the BMSCs. In the case of graft versus host disease, an effective amount of BMSCs is an amount that inhibits or decreases the number of activated T cells present in the transplant.

An effective amount of BMSCs can be determined by comparing the number of activated T cells in a recipient or in a transplant prior to the administration of BMSCs thereto, with the number of activated T cells present in the recipient or transplant following the administration of BMSCs thereto. A decrease, or the absence of an increase, in the number of activated T cells in the recipient of the transplant or in the transplant itself that is associated with the administration of BMSCs thereto, indicates that the number of BMSCs administered is a therapeutic effective amount of BMSCs.

In yet another embodiment of the present invention, the BMSCs can be used as a gene therapy vehicle for the expression of an exogenous gene in a mammal. The benefit of using BMSCs as a vehicle for gene therapy over the presently used cells is based on the novel discovery that BMSCs can survive for longer periods of time when compared with cells presently used in the art for gene therapy applications.

Genetic Modification

The cells of the present invention can be genetically modified by having exogenous genetic material introduced into the BMSCs, to produce factors such as trophic factors, growth factors, cytokines, neurotrophins, and the like. The secreted factors are benefical to neighboring cells. The factors include, but are not limited to, LIF, brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and TGFβ-3.

In another aspect, BMSCs may be genetically modified to express a gene such as HSV-Thymidine Kinase or Green Fluorescent Protein (GFP) which could be used for their removal by treatment with Gancyclovir or separation on a Flow Cytometer respectively.

In addition to genetically modifying BMSCs, the present invention encompasses genetically modified NSCs. Genetically modified NSCs can be used to replace cells that are defective in an individual. The invention may also be used to express desired proteins that are secreted. That is, NSCs can be isolated, introduced with a gene for a desired protein and introduced into an individual within whom the desired protein would be produced and exert or otherwise yield a therapeutic effect. This aspect of the invention relates to gene therapy in which therapeutic proteins are administered to an individual by way of introducing a genetically modified NSC into an individual. The genetically modified NSCs are cultured using the methods disclosed herein, isolated and implanted into an individual who will benefit when the protein is expressed and secreted by the NSC in the body.

According to the present invention, gene constructs which comprise nucleotide sequences that encode heterologous proteins are introduced into the NSCs. That is, the cells are genetically altered to introduce a gene whose expression has therapeutic effect in the individual. According to some aspects of the invention, NSCs from an individual or from another individual or from a non-human animal may be genetically altered to replace a defective gene and/or to introduce a gene whose expression has therapeutic effect in the individual.

In all cases in which a gene construct is transfected into a cell, the heterologous gene is operably linked to regulatory sequences required to achieve expression of the gene in the cell. Such regulatory sequences include a promoter and a polyadenylation signal.

The gene construct is preferably provided as an expression vector that includes the coding sequence for a heterologous protein operably linked to essential regulatory sequences such that when the vector is transfected into the cell, the coding sequence will be expressed by the cell. The coding sequence is operably linked to the regulatory elements necessary for expression of that sequence in the cells. The nucleotide sequence that encodes the protein may be cDNA, genomic DNA, synthesized DNA or a hybrid thereof or an RNA molecule such as mRNA.

The gene construct includes the nucleotide sequence encoding the beneficial protein operably linked to the regulatory elements and may remain present in the cell as a functioning cytoplasmic molecule, a functioning episomal molecule or it may integrate into the cell's chromosomal DNA. Exogenous genetic material may be introduced into cells where it remains as separate genetic material in the form of a plasmid. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Alternatively, RNA may be introduced into the cell.

The regulatory elements for gene expression include: a promoter, an initiation codon, a stop codon, and a polyadenylation signal. It is preferred that these elements be operable in the cells of the present invention. Moreover, it is preferred that these elements be operably linked to the nucleotide sequence that encodes the protein such that the nucleotide sequence can be expressed in the cells and thus the protein can be produced. Initiation codons and stop codon are generally considered to be part of a nucleotide sequence that encodes the protein. However, it is preferred that these elements are functional in the cells. Similarly, promoters and polyadenylation signals used must be functional within the cells of the present invention. Examples of promoters useful to practice the present invention include but are not limited to promoters that are active in many cells such as the cytomegalovirus promoter, SV40 promoters and retroviral promoters. Other examples of promoters useful to practice the present invention include but are not limited to tissue-specific promoters, i.e. promoters that function in some tissues but not in others; also, promoters of genes normally expressed in the cells with or without specific or general enhancer sequences. In some embodiments, promoters are used which constitutively express genes in the cells with or without enhancer sequences. Enhancer sequences are provided in such embodiments when appropriate or desirable.

The cells of the present invention can be transfected using well known techniques readily available to those having ordinary skill in the art. Exogenous genes may be introduced into the cells by standard methods are employed for introducing gene constructs into cell which will express the proteins encoded by the genes. In some embodiments, cells are transfected by calcium phosphate precipitation transfection, DEAE dextran transfection, electroporation, microinjection, liposome-mediated transfer, chemical-mediated transfer, ligand mediated transfer or recombinant viral vector transfer.

In some embodiments, recombinant adenovirus vectors are used to introduce DNA with desired sequences into the cell. In some embodiments, recombinant retrovirus vectors are used to introduce DNA with desired sequences into the cells. In some embodiments, standard CaPO₄, DEAE dextran or lipid carrier mediated transfection techniques are employed to incorporate desired DNA into dividing cells. Standard antibiotic resistance selection techniques can be used to identify and select transfected cells. In some embodiments, DNA is introduced directly into cells by microinjection. Similarly, well-known electroporation or particle bombardment techniques can be used to introduce foreign DNA into the cells. A second gene is usually co-transfected or linked to the therapeutic gene. The second gene is frequently a selectable antibiotic-resistance gene. Transfected cells can be selected by growing the cells in an antibiotic that will kill cells that do not take up the selectable gene. In most cases where the two genes are unlinked and co-transfected, the cells that survive the antibiotic treatment have both genes in them and express both of them.

Unless otherwise stated, genetic manipulations are performed as described in Sambrook et al. (2002, Molecular Cloning: A Laboratory Manual, Cold Spring Harbor Laboratory, New York). The present invention encompasses genetically modified cells, which have been engineered to express an exogenous gene. The exogenous gene can, for example, be an exogenous version of an endogenous gene (for example, a wild type version of the same gene can be used to replace a defective allele comprising a mutation). The exogenous gene is usually but not necessarily covalently linked with (i.e., “fused with”) one or more additional genes. Exemplary “additional” genes include a gene useful for “positive” selection to select cells that have incorporated the exogenous gene, and a gene useful for “negative” selection to select cells that have incorporated the exogenous gene into the same chromosomal locus as the endogenous gene or both.

It should be understood that the methods described herein may be carried out in a number of ways and with various modifications and permutations thereof that are well known in the art. It may also be appreciated that any theories set forth as to modes of action or interactions between cell types should not be construed as limiting this invention in any manner, but are presented such that the methods of the invention can be more fully understood.

The following examples further illustrate aspects of the present invention. However, they are in no way a limitation of the teachings or disclosure of the present invention as set forth herein.

### EXAMPLES

Stem cells have a multitude of potential clinical applications in the repair and replacement of tissues damaged by disease or trauma. The examples herein involve the evaluation of the immunogenicity of NSCs, by characterizing the T cell response to allogeneic NSCs. It has been shown that these cells are recognized by T cells and elicit a proliferative response. The present examples address whether BMSCs can inhibit T cell activation to NSCs in vitro and whether BMSCs can extend NSC survival in vivo. In addition, the present disclosure demonstrates the use of BMSCs to create a localized area of immunosuppression or immune privilege which would aid allogeneic stem cell engraftment.

### Example 1

### Characterization of Neural Stem Cells (NSCs)

Isolation and Culture of Human Fetal Neural Stem Cells:

Human fetal brain tissue can be purchased from Advanced Bioscience Resources (Alameda, Calif.). The tissue is washed with PBS/antibiotics and the meninges are removed prior to using the desired brain tissue. The remaining tissue is teased apart with a pair of forceps and further dissociated by trituration with a Pasteur pipette. Cells are then pelleted by centrifugation at 1000 rpm for 5 minutes at room temperature. The cell pellet is resuspended in 10 ml of NSC growth medium (DMEM/F12, 8 mM glucose, glutamine, 20 mM sodium bicarbonate, 15 mM HEPES, 8 μg/ml Heparin, N2 supplement, 10 ng/ml bFGF, 20 ng/ml EGF). The cells are plated on a coated (15 μg/ml Polyornithine overnight followed by 10 μg/ml human Fibronectin for greater than 4 h) T-25 cm² flask with vented cap and grown in a 5% CO₂ incubator at 37° C. Cells grown with Leukemia Inhibitory Factor (LIF) are plated in the complete growth medium with 10 ng/ml LIF after growing them initially (1-2 passages) in the presence of bFGF and EGF alone. Cultures are fed every other day by replacing 50% of the medium with fresh complete growth medium. Cells are passaged by trypsinization with 0.05% Trypsin-EDTA in PBS for 2-3 minutes followed by addition of soybean trypsin inhibitor to inactivate the trypsin. The cells are pelleted at 1200 rpm for 5 minutes at room temperature, resuspended in growth medium, and plated at 1.0-1.25×10⁵ cells/cm² on coated flasks. Cells are cryopreserved in 10% DMSO+90% complete growth medium.

NSCs Express MHC Class I Antigens:

NSCs were prepared from human fetal tissue using methods described herein and were cultured for about 13 passages. The cells were evaluated for immunologically relevant cell membrane molecules using standard flow cytometry techniques. Briefly, cells are washed in PBS containing 5% FBS and blocked with mouse immunoglobulin prior to staining with fluorochrome tagged monoclonal antibodies. Background staining is determined by incubating cells with isotype-matched fluorochrome-labeled immunoglobulins. About 50,000 cells are acquired for analysis on a Becton Dickinson FACSCaliber flow cytometer using Cell Quest acquisition software. Because BMSCs/NSCs and hematopoietic cells differ greatly in their size and light scatter properties, it is preferable to acquire the cells on the cytometer separately for each cell type. The gate coordinates were determined by spiking BMSCs with PBMCs. Data are displayed as histograms or dot blots.

It was observed that the population of NSCs did not express hematopoietic markers (CD45, CD14, CD34), costimulatory molecules (CD80, CD86), or MHC class II molecules. However, the NSCs did express the stem cell marker, CD133, as well as MHC class I antigens. The expression of class I molecules usually indicates that the cells would be recognized by alloreactive T cells and would be rejected if transplanted into an immunocompetent allogeneic recipient.

NSCs Stimulate Proliferation of Allogeneic T Cells

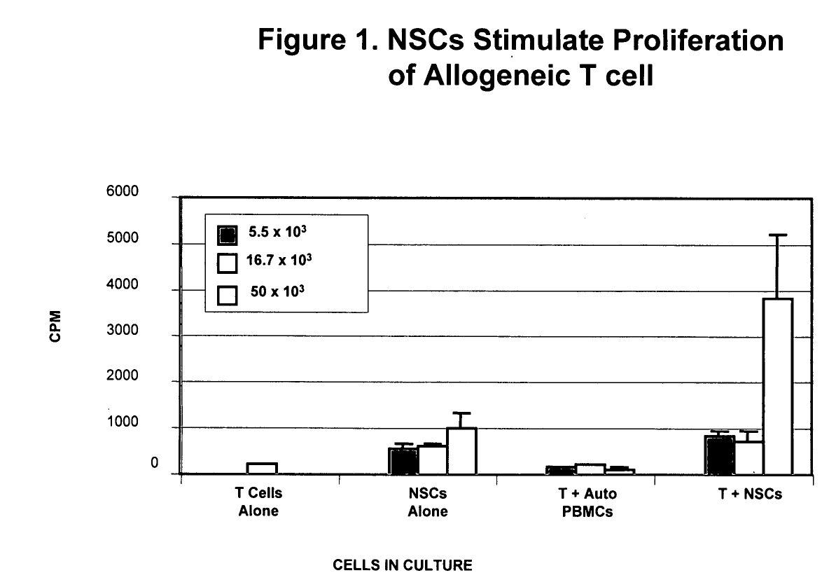

The immunogenicity of NSCs was evaluated by one-way mixed lymphocyte reaction (MLR) using T cells as responding cells and irradiated NSCs as stimulator cells. The MLR, which measures the T cell proliferative response to alloantigens, is predictive for survival of allogeneic cells in vivo. NSCs were prepared from human fetal tissue as described elsewhere herein and cultured for about 13 passages. Starting with a high dose of about 5×10⁴ cells/well, the NSCs were titrated down in 3-fold decrements as stimulator cells. Purified T cells (2×10⁵ cells/well) from an unrelated donor were prepared as responder cells. Autologous PBMCs were used as control stimulator cells. As shown in FIG. 1, NSCs stimulated a significant degree of T cell proliferation compared to autologous PBMCs, which did not stimulate a significant amount of T cell proliferation even at the highest cell dose (P<0.05, Student's t test). These results demonstrate that allogeneic NSCs are recognized by T cells and induce a functional immune response.

### Example 2

### Characterization of Bone Marrow Stromal Cells (BMSCs)

BMSCs were produced by methods known in the art. For example, human bone marrow can be purchased from AllCells LLC (Berkeley, Calif.) or collected via needle aspirate from a donor. The cells are spun over a Hespan density gradient at 800×g for 30 min, and cells at the interface are plated at 1.62×10⁵/cm² in T-185 flasks using Dulbecco's Modified Eagles Medium (DMEM) supplemented with 10% FBS. Nonadherent cells are removed from the population of cells following a period of culturing, and the medium is changed every 3-4 days until the cells become confluent (P0), which typically occurs after about 2-3 weeks of culturing. Adherent cells are collected by trypsinization (0.05% trypsin) and replated at a seeding density of 5000 cells/cm² in T185 flasks if further expansion is desired. Culture medium is changed every 3-4 days until the cells become confluent (usually 1 week) and are passaged by trypsinization. The cells were tested for purity using specific markers by flow cytometry. BMSCs are usually passaged 2-4 times prior to being used for experimentation or cryopreserved in liquid nitrogen.

Rat BMSCs can be isolated using methods known in the art. For example, hind legs are removed and the femur is dissociated from the tibia. Sterile bone cutters are used to remove the distal end of the femur and a syringe fitted with an 18 gauge needle is used to flush the marrow plugs into sterile media, for example Alpha Modified Eagles Medium supplemented with 10% fetal bovine serum and penicillin/streptomycin. Marrow plugs are resuspended into a single cell suspension and the suspension is passed through a 100 micron cell strainer to remove debris. The cells are washed and resuspended in culture medium. Cells are plated in T-185 flasks at a density of about 9×10⁵/cm². Flasks are cultured at 37° C. in a humidified atmosphere containing 5% CO₂. After 2 days, the media containing nonadherent cells is aspirated and replaced with fresh media. Cultures are fed every 3-4 days until adherent stromal cells become confluent (approximately 14 days). These P0 cells are trypsinized (0.25% trypsin) for 5 minutes at 37° C., fresh media is added to inactivate trypsin, and the cells are washed and replated at 1.08×10⁴/cm². Generally, cells are passaged every week as they become confluent. By passage 3, there is less than 1% contamination by hematopoetic cells, assessed by flow cytometry using antibodies to CD45, T cells (OX-52), macrophages (CD11b/c), and B cells (anti-kappa light chain). BMSCs stain positive for Thy-1 (CD90) and MHC class I (OX-18); they are negative for MHC class II antigens.

BMSC Phenotype

Human BMSCs were produced from bone marrow as described elsewhere herein and cultured for 2 passages. They were stained with a variety of fluorochrome-labeled monoclonal antibodies and evaluated for expression of cell surface markers by flow cytometry. BMSCs express CD90, CD105, and MHC class I markers, whereas they do not express hematopoietic markers including CD45, CD3, CD34, CD19, and CD14. They also do not express MHC class II molecules.

BMSCs do not Stimulate Allogeneic T Cell Proliferation

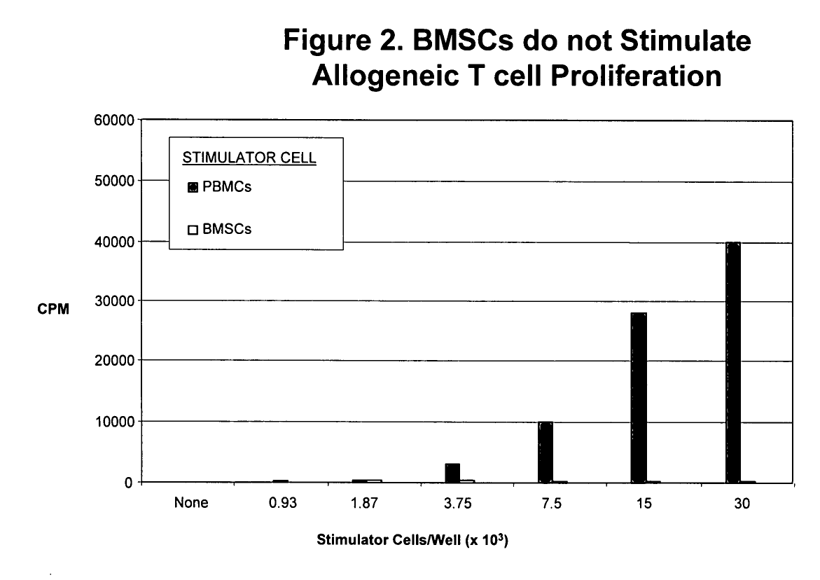

The immunogenicity of BMSCs was evaluated by one-way mixed lymphocyte reaction (MLR) using T cells as responding cells and irradiated BMSCs as stimulator cells. The results demonstrate that T cells do not proliferate to allogeneic BMSCs, but they respond vigorously to allogeneic PBMCs (FIG. 2).

Human MLR Assays:

The immunogenicity of BMSCs was evaluated by mixed lymphocyte reaction (MLR) using T cells as responding cells and allogeneic PBMCs as stimulator cells. T cells were purified from leukopheresis packs (AllCells,LLC, Berkeley, Calif.; Poietics, Rockville, Md.) by negative selection using mouse monoclonal antibodies (Serotec, Raleigh, N.C.) specific for monocytes (CD14), B cells (CD19), MHC class II, and NK cells (CD56) to label non-T cells for removal using magnetic beads coated with monoclonal anti-mouse IgG antibody (Dynal Biotech, Inc, Lake Success, N.Y.). The remaining population of cells after depletion was typically greater than 85% CD3 positive by flow cytometry analysis. T cells were suspended in culture medium: Iscove's Modified Dulbecco's Medium (IMDM) supplemented with sodium pyruvate, non-essential amino acids, 2-mercaptoethanol, antibiotic/antimycotic, and 5% human AB serum (all supplements were obtained from Gibco, Carlsbad, Calif. except human AB serum which was obtained from PelFreez, Brown Deer, Wis.). T cells were seeded into microtiter wells (2×10⁵/well) in 96-well low evaporation flat-bottom plates (BD Falcon, Franklin Lakes, N.J.) with allogeneic stimulator cells. Stimulator cells were irradiated with 5000 rads gamma irradiation using a cesium source (Isomedix Gammator B, Parsippany, N.Y.) prior to being plated at the numbers necessary for the experiment (usually titrated decrementally from a high dose of 5×10⁴ cells/well). Cultures were set up in triplicate wells per treatment. T cell proliferation to alloantigens was determined by pulsing the cultures with ³H-thymidine on the sixth day of culture. The cells were harvested onto filtermats 16 hours later using a 96 well cell harvester (Skatron, Molecular Devices Corp, Sunnyvale, Calif.), and the cells on the filtermats were counted using a Microbeta scintillation counter (Perkin Elmer, Turku, Finland).

Rat MLR Assays:

These assays are set up in similar fashion to the human MLRs. Briefly, responder cells are produced by harvesting cervical plus mesenteric lymph node cells (LNCs). The responder cells are dissociated into a single-cell suspension by grinding them with a syringe plunger against a cell strainer (BD Falcon) in a 6-well plate. The responder cells are suspended in culture medium similar to human MLR medium with the exception that the serum is 10% FBS (HyClone, Logan, Utah). LNCs are seeded into microtiter wells (4×10⁵/well) with allogeneic stimulator cells at the numbers necessary for the experiment. Stimulator cells are irradiated (5000 rads) prior to being plated. Cultures are set up using triplicate wells per treatment. T cell proliferation to alloantigens are assessed as described elsewhere herein.

Immunogenicity Experiments

The one-way MLR assay can be used to evaluate T cell proliferation to allogeneic BMSCs. Briefly, T cells (2×10⁵/well) are cultured with irradiated (5000 rads gamma radiation) allogeneic BMSCs, autologous PBMCs, or allogeneic PBMCs (30,000 cells/well) in 96 well microtiter culture plates. The BMSCs are obtained using methods disclosed herein. T cells were purified from PBMCs obtained from four different donors using methods known in the art. T cell enrichment can be achieved by negative selection, using magnetic beads (Dynal, Inc) to remove non-T cells. Mouse monoclonal antibodies (mAbs) specific for macrophages/monocytes/dendritic cells (anti-CD14), B cells (anti-CD19), NK cells (anti-CD56), and MHC class II antigens (anti-DR) are used to label these cells. Magnetic particles coated with goat anti-mouse IgG antibody are used to pull the cells out in a magnetic field. The resulting cell population is typically greater than 90% T cells by flow cytometry analysis using fluoresceinated anti-CD3 mAb to detect T cells. Cell culture medium used can be Iscove's Modified Dulbecco's Medium (IMDM) supplemented with 5% human AB serum, non-essential amino acids, sodium pyruvate, pen-strep/fungizone, and 2-mercaptoethanol. The cultures are incubated in a humidified atmosphere of 5% CO₂ at 37° C. for 6 days, pulsed with ³H-thymidine (1 μCi/well) for 16 hrs, and the cells are harvested on day 7 using an automated 96 well cell harvester. The incorporated radioactivity is determined by scintillation counting and the results are reported as counts per minute (cpm).

BMSCs do not Induce Expression of the Activation Molecule CD25 on Allogeneic T Cells

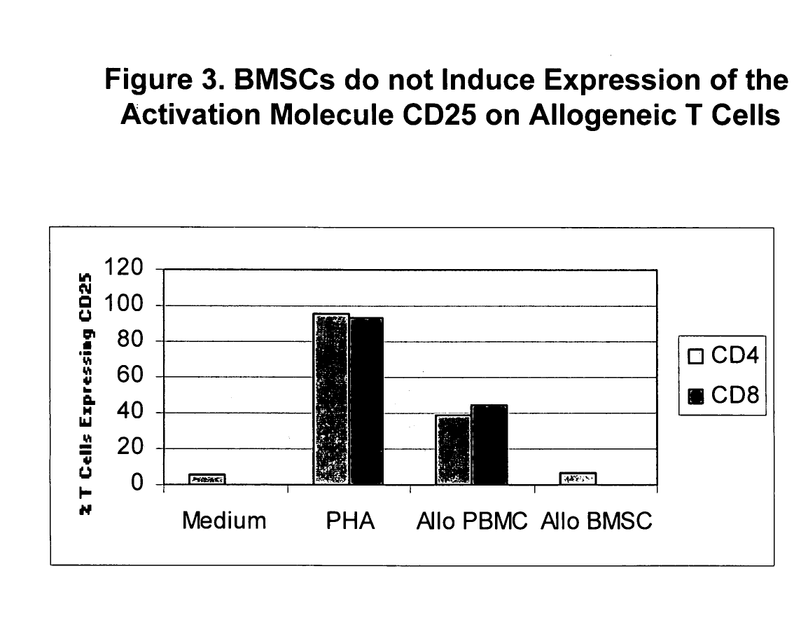

Purified human T cells were cultured in culture medium in the presence of the following: 1) T cell mitogen PHA, 2) allogeneic PBMCs, or 3) allogeneic BMSCs. After seven days, T cells were harvested from the cultures and dually stained with monoclonal antibodies specific for T cell subsets, CD4 and CD8 (FITC-labeled mAbs), as well as the T cell activation marker, CD25 (APC-labeled mAb). Background staining was standardized by using appropriately labeled isotype control immunoglobulins. The results demonstrated that both T cell subsets were activated by PHA and allogeneic PBMCs, but not by allogeneic BMSCs (FIG. 3).

BMSCs Suppress T Cell Proliferation.

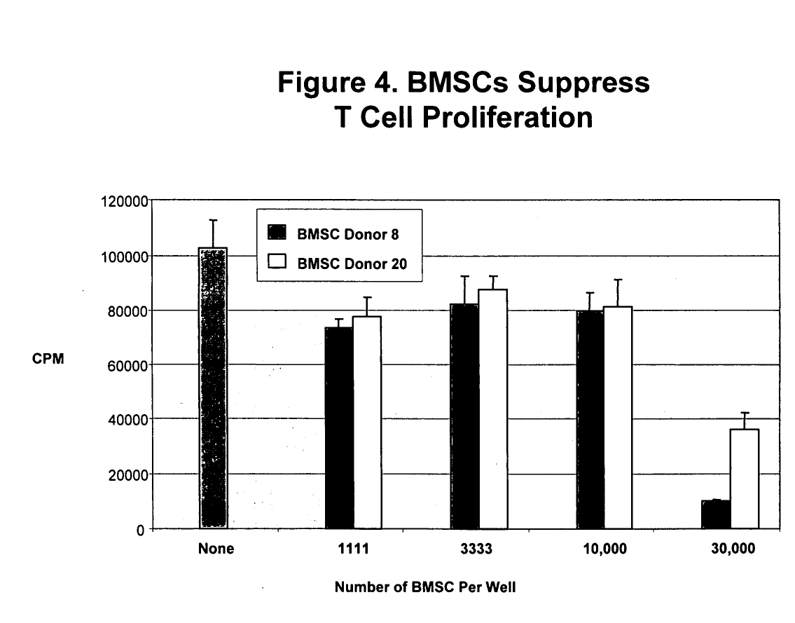

To determine whether BMSCs suppressed alloreactive T cells, BMSCs were titrated into MLR cultures consisting of T cells (2×10⁵ cells/well) plus allogeneic irradiated PBMCs (1×10⁵ cells/well). BMSCs from 2 different donors were added to MLR cultures at the initiation of the culture period. The results showed that T cell proliferation was suppressed by 62% (Donor 8) and 90% (Donor 20) at the highest dose of BMSCs (3×10⁴ cells/well) (FIG. 4). Based on these results, it is believed that a ratio of approximately 1 BMSC to 7 responder T cells should be sufficient for significant suppression to occur.

The results demonstrated herein that NSCs induced allogeneic T cell proliferation in an MLR. Without wishing to be bound to any particular theory, these cells may be rejected by the hose if they were transplanted in an allogeneic setting. Even though BMSCs expressed a similar constellation of cell surface markers as compared with NSCs, it was observed that BMSCs did not activate T cells. Based on the results herein, it is believed that BMSC possess immunosuppressive properties.

Suppression Experiments

BMSCs are added to one-way MLR assays to determine whether they could suppress alloreactive T cell proliferation. Briefly, BMSCs are irradiated (5000 R) and added to one-way MLR cultures comprising purified T cells (responder cells) and irradiated allogeneic PBMCs (stimulator cells). Purification of T cells and culture conditions for the MLR are described elsewhere herein. BMSCs were added to MLR cultures between different combination of T cells and PBMCs at a dose of 30,000 cells/well. Results can be shown as percent suppression of the base MLR response to which no BMSCs were added. Suppression was determined by comparing the response containing the test cells to the control MLR according to the following formula:
Percent Suppression=[1−(cpm of MLR culture containing test cells/cpm control MLR culture)]×100.

### Example 3

### BMSC in Transplantation

The following experiments are designed to explore BMSC-mediated immunoprotection of stem cells from rejection. Any stem cell can be used, but for the following experiments, human NSCs are used as a model stem cell due to availability and previous data indicating that they are immunogenic.

In order to address immunologic responses to NSCs in vitro, T cell responses to NSCs are compared to allogeneic PBMCs (positive control) and BMSCs with the intention of learning what type of T cell response each cell type elicits. Without wishing to be bound by any particular theory, the results from these experiments can provide insight into whether T cells are activated by proliferation and CD25 marker expression, and whether the T cells are CD4 or CD8 positive. Based on the cytokine profile, it can be determined whether the cells are Th1 or Th2-type T cells.

A second group of experiments are designed to determine whether BMSCs can prevent T cell activation on NSCs in vitro and whether BMSCs effect cytokine production during the course of immunosuppression. Based on the present disclosure, it is believed that BMSCs added to cultures of T cells and allogeneic NSCs can suppress T cell proliferation in a genetically unrestricted manner.

Another set of experiments are designed to assess the ability of BMSCs to protect NSCs from graft rejection in vivo. By way of an example herein, a xenogeneic model involving implanting human NSCs into the livers of recipient rats with rat fibroblasts (control) or with rat BMSCs is used. However, based on present disclosure, a skill artisan would be able to use an allogeneic model. The xenogeneic model encompasses the following criteria:

1) human NSCs as the donor cell;

2) rat BMSCs because human BMSCs have been shown to be non-immunogenic in terms of inducing allogeneic T cell proliferation (McIntosh et al., 2000 Graft 3:324);

3) the liver was chosen as the site of implantation due to the ease of administration of cells through the portal vein, the accessibility of the injected cells to the immune system, and the ability to recover injected cells. Without wishing to be bound to any particular theory, BMSCs injected intraportally may become lodged in the liver. Further, since NSCs are approximately the same size as BMSCs, NSCs may also become trapped in the liver after portal delivery. The use of human NSCs as the only human cell in this model enables the assessment of the engraftment using a PCR technique specific for human Alu DNA sequences. The model described herein also can be used to determine whether the NSCs and BMSCs induce a T cell response in the lymph nodes of recipient animals by using these cells in one-way MLR assays.

### Example 4

### Characterize the T cell response to Allogeneic NSCs and BMSCs

The T cell response to NSCs are characterized from three different donors, and the response is compared to responses obtained against BMSCs. Unfractionated freshly thawed PBMCs are used as controls. Each of these populations of cells are evaluated for: 1) immunologically relevant cell surface markers, 2) the ability to stimulate allogeneic T cells, and 3) a T cell response for expression of activation markers and cytokines. Data generated from these experiments provide insight into the characteristics determining immunogenicity of each of these populations of cells.

Characterization of Immunologically Relevant Cell Surface Markers by Flow Cytometry

NSCs and BMSCs are evaluated at the lowest passage possible needed to obtain sufficient numbers of cells for these experiments. These cells are harvested from ongoing cultures in T-185 flasks (5×10⁶ cells/flask) using 0.05% trypsin. Freshly thawed PBMCs are used as controls. The cells are characterized for expression of MHC antigens (class I and class II) and for costimulatory molecules (CD40, CD54, CD80, CD86) by flow cytometry. Briefly, cells are washed in PBS containing 5% FBS and blocked with mouse immunoglobulin prior to staining with fluorochrome tagged monoclonal antibodies. Background staining is determined by incubating cells with isotype-matched fluorochrome-labeled immunoglobulins. About 50,000 cells are used for analysis on a Becton Dickinson FACSCaliber flow cytometer using Cell Quest acquisition software. Because BMSCs/NSCs and hematopoietic cells differ greatly in their size and light scatter properties, it may be necessary to acquire the cells on the cytometer separately for each cell type. The gate coordinates are determined by spiking BMSCs with PBMCs. That data are displayed as histograms or dot blots.

Determine T Cell Proliferative Responses to Allogeneic NSCs and BMSCs by the MLR Assay.

One-way MLR assays are used to evaluate a functional response by T cells to allogeneic stimulator cells. The MLR assays are designed to compare purified T cells to unfractionated PBMCs as responder cells in order to assess direct versus indirect T cell activation (i.e., whether stem cells behave as antigen presenting cells (APCs) by directly activating T cells, or whether stem cells activate T cells indirectly through professional APCs (macrophages, dendritic cells) that pick up their alloantigens).

Mixed lymphocyte cultures are set up using unfractionated PBMCs or purified T cells (both from the same donor) as responder cells and NSCs or BMSCs as stimulator cells. Briefly, T cells (2×10⁵/well) are cultured with irradiated (5000 rads gamma radiation) allogeneic BMSCs, autologous PBMCs, or allogeneic PBMCs (30,000 cells/well) in 96 well microtiter culture plates. The BMSCs are obtained from different donors using methods disclosed herein. T cells are purified from PBMCs obtained from methods known in the art. T cell enrichment is achieved by negative selection, using magnetic beads (Dynal, Inc) to remove non-T cells. Mouse monoclonal antibodies (mAbs) specific for macrophages/monocytes/dendritic cells (anti-CD14), B cells (anti-CD19), NK cells (anti-CD56), and MHC class II antigens (anti-DR) are used to label these cells. Magnetic particles coated with goat anti-mouse IgG antibody are used to pull the cells out in a magnetic field. The resulting cell population are typically greater than 90% T cells by flow cytometry analysis using fluoresceinated anti-CD3 mAb to detect T cells. The cells are cultured in Iscove's Modified Dulbecco's Medium (IMDM) supplemented with 5% human AB serum, non-essential amino acids, sodium pyruvate, pen-strep/fungizone, and 2-mercaptoethanol. The cultures are incubated in a humidified atmosphere of 5% CO₂ at 37° C. for six days, pulsed with ³H-thymidine (1 μCi/well) for 16 hours, and the cells are harvested on day seven using an automated 96 well cell harvester. The incorporated radioactivity is determined by scintillation counting and the results are reported as counts per minute (cpm).

In order for a test cell to be deemed immunogenic, three requirements must be met:

1) the test cell must induce a T cell proliferative response that is at least 750 cpm above the autologous response;

2) the Stimulation Index (T+test cell cpm/T+autologous cell cpm) must be greater than or equal to 3.0; and

3) there must be a statistically significant difference between T+autologous PBMCs and T+test cells (P<0.05, Student's t test).

To rule out genetic factors (i.e., MHC similarities) as the cause of non-responsiveness, each test cell is cultured with at least 2 (preferably 3) different T cell donors. If any of the donors produced a positive response as described above, the test population is considered immunogenic.

NSCs are expected to stimulate significant T cell proliferation, but not as high as that seen to allogeneic PBMCs which contain large numbers of professional APCs. BMSCs should not stimulate T cell proliferation since they are immunosuppressive. It is believed that if NSCs can function directly as APCs or if there are APC-populations present within the NSCs, these cells should stimulate proliferation of purified T responder cells. T cells within the APC-containing PBMC responder population may proliferate more vigorously due to indirect antigen presentation. The temporal kinetics of the latter response may be shifted toward longer time periods for optimal proliferation since alloantigens are needed to be taken up by APCs in the PBMC population, processed, and presented to stimulate T cells.

Determine CD25 Up-Regulation on T Cell Subsets in Response to Allogeneic NSCs and BMSCs by Flow Cytometry.

CD25 (IL-2 receptor) is up-regulated on activated T cells. In order to define an additional parameter other than proliferation as a marker of T cell activation, and to further assess activation at the subpopulation level, CD25 can be analyzed on CD4- and CD8-positive T cells after culture with NSCs and BMSCs.

NSCs and BMSCs used in the following experiments are harvested from T-185 flask cultures as described elsewhere herein. Bulk MLR cultures are set up in 6 well plates, in triplicate for harvesting at 3 time points. Purified T cells (14×10⁶ cells/well) are cultured in medium, with autologous PBMCs (7×10⁶/well), with allogeneic PBMCs (7×10⁶/well), with NSCs (2×10⁶ cells/well), or with BMSCs (2×10⁶ cells/well). NSCs and BMSCs are also cultured in medium alone (2×10⁶ cells/well) as additional controls. All stimulator cells are irradiated with 5000 rads prior to culture. The cultures are terminated at 1, 4 and 7 days, and supernatants are collected for cytokine analysis. Nonadherent T cells are collected by gently washing the cultures and the cells are analyzed by 3-color flow cytometry for CD3, CD4 or CD8, and CD25 expression using APC, FITC, and PE fluorochrome-conjugated antibodies, respectively. The expression of CD25 is compared to the baseline culture of T cells plus autologous PBMCs.

Without wishing to be bound by any particular theory, CD25 should be up-regulated on both CD4- and CD8-positive T cells in response to PBMCs since both allogeneic MHC class I and II molecules are present. Since NSCs express MHC class I but not MHC class II, CD25 may appear on activated CD8+ T cells but is not expected to be observably present on CD4+ T cells. In addition, it is not expect to observe CD25 expression on T cells that are cultured with BMSCs.

Characterize T Cell Cytokine Responses to Allogeneic NSCs, BMSCs, and PBMCs.

To further characterize the T cell response to allogeneic stem cells, the cytokine response to NSCs and BMSCs can be assessed (PBMCs is used as controls). A Th1 type response should secrete IFN-γ and IL-2, whereas a Th2 response favors IL-4 and IL-10 (Mossmann et al., 1989, Annu. Rev. Immunol. 7:145). If the response is polarized toward a classic Th1 rejection response, cytokine transduction strategies can be employed in order to down-regulate this response by inducing a Th2 response (i.e., utilizing IL-4 cytokines, or down-regulating the response using IL-10).

Supernatants are harvested from bulk-cultures of cells in 6-well plates as described elsewhere herein. At least 4 ml of supernatant are harvested per well when the cells are collected for flow cytometry, on days 1, 4 and 7. Cytokines are evaluated by RayBio® Cytokine Antibody Array (RayBiotech Inc., Norcross, Ga.) that detects multiple cytokines in biological samples. A customized array can be used to detect IL-2, INFγ, IL-4, and IL-10 in addition to G-CSF, GM-CSF, Interleukins-1α, -3, -5, -6, -7, -8, -12, -13, -15, MCPs-1,2, and 3, MIG, RANTES, TNF-α and β, and TGF-β1. Further quantitative measurements of cytokines of interest can be performed by ELISA.

Not wishing to be bound by any particular theory, PBMC and NSC stimulators should elicit a classic Th1 response with high levels of IL-2 and IFN-γ.

### Example 5

### Determine Immunosuppressive Effects of BMSCs on T Cell Responses to Allogeneic NSCs in Vitro

The following in vitro studies address whether immunosuppressive BMSCs can prevent T cell proliferation against NSCs and whether genetic matching of the BMSCs to the responding T cells makes a difference in the suppression. In addition, the experiments herein includes characterizing cytokines produced during the course of suppression as measured by using cytokine arrays. Cytokines of interest include, but are not limited to cytokines which would: 1) define transition from a Th1 to Th2-type T cell response; 2) enhance MHC expression on stem cells, and 3) have suppressive effects.

Determine Suppressive Effect of BMSCs on T Cell Proliferation in the MLR Response.

The present disclosure demonstrates that BMSCs can suppress T cell alloreactivity against NSCs in vitro. BMSCs from a minimum of 3 different donors are used to evaluate the effects of BMSCs on T cell proliferation. To assess whether alloreactivity mediated against the BMSCs is beneficial for suppression, the effect of HLA matching by comparing BMSCs matched (autologous) to the T cells are compared to using unmatched BMSCs.

To investigate suppression of primary MLRs, T cells (2×10⁵/well) are cultured with allogeneic PBMCs (5×10⁴/well) or NSCs (5×10⁴/well) in microtiter plates as described elsewhere herein. BMSCs (autologous to T cells) are added to the cultures starting at 5×10⁴ cells/well, and they are titrated in 2-fold decrements down to 3,125 cells/well to generate a dose-response curve. Control cultures receive no BMSCs. Percent suppression are determined by comparing the MLR response in the absence of BMSCs to the response in the presence of BMSCs. Statistically significant suppression is determined by using Student's t test.

Based on the present disclosure, BMSCs suppress primary responses. BMSCs from two different donors were observed to suppress a primary MLR response. Suppression of ongoing and secondary MLR responses can also be examined using the methods disclosed herein.

Characterize the Cytokine Profile in Suppressed MLR Cultures.

Characterization of cytokines produced during the course of suppression is achieved by comparing the cytokine profile to that of the base MLR alone. Results from the cytokine profile can be used to determine the mechanism of suppression by BMSCs.

Bulk MLR cultures in 6-well plates containing T cells (14×10⁶/well) and allogeneic NSCs or PBMCs (2×10⁶/well) are set up in the presence and absence of BMSCs (2×10⁶/well). Stimulator cells are irradiated with 5000 rads prior to culturing. At days 1, 4 and 7, cultures are terminated. Supernatants are harvested and tested for 25 cytokines using the RayBio® Cytokine Antibody Array (RayBiotech Inc., Norcross, Ga.). Comparisons are made between T+NSC and T+NSC+BMSC, and between T+PBMC and T+PBMC+BMSC to evaluate the effect of BMSC suppression on the cytokine response.

Without wising to be bound by any particular theory, it is believed that BMSCs can induce shifts in cytokines that are normally produced in an MLR response.

### Example 6

### Determine whether BMSCs can Extend Survival of Transplanted NSCs in Rats

Experiments disclosed herein address the contribution of BMSCs to the survival of transplanted NSCs in a mammal. It is believed that if BMSCs are suppressive for alloreactive T cell responses in vitro, they could also suppress these responses in vivo. Therefore, if BMSCs were implanted in sufficient numbers in a tissue, they may create an area of “immune privilege” whereby allogeneic cells could be implanted without inducing a rejection response. As proof of principle of the immunoprotection concept, the following experiments involve implanting human NSCs in the livers of recipient rats, with control rat fibroblasts or rat BMSCs, and quantitating their survival over a period of one month using human-specific Alu PCR. The following xenogeneic model was chosen due to issues of stem cell availability and a high degree of certainty that rat BMSCs suppress the xenogeneic responses between rat T cells and human stimulator cells.

Allogeneic rat BMSCs are used in these experiments so as to replicate a desirable clinical path using third party cells. The liver was chosen as the site of implantation due to ease of delivery via the portal vein, access to the immune system, and ability to recover the injected cells. It has been shown that MSCs administered by this route become trapped in the liver and are detectable for at least one week. Lymph nodes are recovered from treated rats and analyzed for sensitization to each of the cell types injected (rat fibroblasts, rat BMSCs, and human NSCs) by one-way MLR.

Suppression of Xenogeneic Rat Versus Human MLR by Rat BMSCs

Xenogeneic MLRs are set up between rat and human cells to evaluate rat BMSC-mediated suppression. Rat BMSCs allogeneic to the recipient are used in these set of experiments, but BMSCs from any source can be used to suppress MLR responses. For example, BMSCs autologous to the recipient can also be used.

Fisher rat lymph nodes (LNs) are harvested, dissociated into a single-cell suspension, and plated in microtiter wells (4×10⁵/well) as responder cells in the MLR. Human PBMCs and NSCs are irradiated and plated at 1×10⁵ per well as stimulator cells. BMSCs and skin fibroblasts from ACI strain rat are titrated into the MLRs at a high dose of 5×10⁴ cells/well and 2-fold decremental doses down to 3,125 cells/well. It should be appreciated that BMSCs and skin fibroblasts can be from any rat strain. Culture conditions for the rat MLR are described elsewhere herein. Suppression is determined by comparing the control MLR (no BMSCs) to MLRs containing fibroblasts or BMSCs. Statistical evaluations are performed using the Student's t test.

Based on the fact that Wistar rat LNCs were observed to proliferate vigorously (>100×10³ cpm) to human PBMCs, it is appreciated that using rat BMSCs in a xengeneic MLR represents a valuable model for assessing the level of suppression BMSCs have in MLRs. Rat BMSCs have been shown to induce unresponsiveness in activated alloreactive T cells (Small et al., 1999 Blood 94:333). Without wishing to be bound by any particular theory, it is believed that rat BMSCs suppress the rat/human MLR. In the event that ACI strain BMSCs do not suppress the xenogeneic MLR, BMSCs derived from other rat strains as well as by human BMSCs can be used in the xenogeneic MLR. It is not expected the rat fibroblasts can suppress the MLR, and therefore fibroblasts are used as a control.

Determine Survival of Transplanted NSCs

The following experiments are designed to assess the survival of NSCs in vivo after administration with control fibroblasts or BMSCs. Based on the present disclosure, a 1:1 ratio of BMSCs to NSCs should be adequate for suppression in vivo in view of a 1:3 ratio of BMSCs to stimulator PBMCs was observed to be sufficient in vitro. Further, it is believed that PBMCs are stronger stimulators of T cells than NSCs, and therefore warranting the 1:1 ratio of BMSCs to NSCs. In addition, a ratio of fewer BMSCs to NSCs can be used for suppression in vivo.

Human NSCs (5×10⁶ cells) mixed with ACI strain rat dermal fibroblasts (5×10⁶ cells) are injected intraportally in a volume of 200 μl into each of 25 Fisher rats. Dermal fibroblasts are produced from skin samples obtained from ACI rats and expanded as described for rat BMSCs elsewhere herein. Another group of 25 rats are injected intraportally with NSCs (5×10⁶ cells/rat) mixed with an equal number of ACI strain rat BMSCs (5×10⁶ cells/rat). Five rats from each group are sacrificed on days 1, 7, 14, 21, and 28 after injection. The livers are removed, snap frozen, and stored at −80° C. until the Alu PCR assay is performed in order to assess the engraftment of the human NSCs.

Human Alu PCR Assay: The number of human NSCs in rat livers can be quantified using an intra-Alu element-based PCR assay described by Walker et al. (2003, Analytical Biochem. 315:122-128). The naturally occurring repetitive Alu sequence present in human DNA allows greater detection sensitivity over single copy sequences/genes. Thus, genomic DNA of human origin is quantified via RealTime PCR for the human-specific Alu repeat sequence. The primers employed for this assay amplify an intra-Alu core repeat sequence of ˜200 bp within the Yb8 Alu subfamily. Use of these primers was reported by Walker et al. (2003, Analytical Biochem. 315:122-128) to allow human DNA specific detection to at least 10 pg (˜1 cell equivalent) in 2 ng of mixed species sample DNA. Genomic DNA is isolated from snap-frozen rat livers using the Puregene DNA Isolation kit (Gentra Systems). Human DNA is quantitated by comparison with an Alu-specific DNA standard curve generated from populations of rat cells spiked (in 10-fold increments) with known numbers of human cells.

Based on the present disclosure, it is believed that BMSCs mediate localized suppression in vivo, and extend the survival of xenogeneic cells in the liver. Thus, a greater number of NSCs from rats that were given BMSCs are recovered than from rats that received NSCs with non-suppressive fibroblasts. The greatest difference between these 2 groups would be expected to occur after 1-2 weeks, when the immune response is activated to the xenogeneic NSCs. A PCR assay can be used to detect NSCs that have survived the transplantation by measuring the human-specific Alu repeat sequence.

Determine T Cell Priming to Injected Cells in Recipient Rats:

These following experiments are designed to determine whether NSCs, fibroblasts, or BMSCs primed allo/xeno-reactive T cells in peripheral lymph nodes of recipient rats. A one-way MLR assay can be used to evaluate such priming.

Cervical and mesenteric lymph nodes (LNs) are removed from 2 groups of 5 rats each (NSCs+fibroblasts versus NSCs+BMSCs) that are sacrificed at the final time point, one month after injection. LNs from the rats are dissociated into a single-cell suspension and used as responder cells in MLR assays with irradiated donor NSCs, fibroblasts, and BMSCs as stimulator cells (5×10⁴ cells/well). Control groups used in these experiments are irradiated syngeneic Fisher strain spleen cells as stimulators (background), LNCs cultured in medium alone, and irradiated stimulator cells cultured in medium alone. Each rat is evaluated separately. The MLR assays are harvested at days 3 and 7 to determine peak response. The mean response to each stimulator population are compared to background responses to syngeneic spleen cells. The Student's t test is used to determine statistically significant differences.

Without wishing to be bound by any particular theory, if the T cells were primed in vivo to allogeneic or xenogeneic cells, they should display an accelerated proliferative response to these cells in an MLR which would peak on day 3 rather than on day 7 (the latter time point is the peak activity observed for primary MLRs). If xenogeneic NSCs primed recipient rats, their T cells should give a secondary response in the MLR assay to NSCs as stimulator cells. In contrast, if BMSCs prevented an immune response to the NSCs, recipient T cells should give a primary MLR response. If BMSCs tolerized recipient T cells to NSCs, they should give reduced responses in an MLR.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of treating a transplant recipient, wherein said transplant is a neural stem cell (NSC), to reduce in said recipient an immune response of effector cells against an alloantigen to said effector cells, the method comprising;
administering to a transplant recipient, bone marrow stromal cells in an amount effective to reduce an immune response of effector cells against an alloantigen to said effector cells, whereby in the transplant recipient said effector cells have a reduced immune response against said alloantigen.

2. The method of claim 1, wherein said effector cells are T cells.

3. The method of claim 2, wherein said T cells are from a donor and said alloantigen is from a recipient.

4. The method of claim 2, wherein said T cells are from a recipient and said alloantigen is from a donor.

5. The method of claim 2, wherein said T cells are present in the transplant.

6. The method of claim 1, wherein prior to said administering to a transplant recipient bone marrow stromal cells, said bone marrow stromal cells have been expanded in culture.

7. The method of claim 1, wherein said effector cells are T cells activated prior to administration of bone marrow stromal cells, and further wherein said immune response is the reactivation of said T cells from the donor.

8. The method of claim 1, wherein said bone marrow stromal cells are administered to the transplant recipient to treat rejection of the transplant by the recipient.

9. The method of claim 1, wherein said bone marrow stromal cells are human bone marrow stromal cells.

10. The method of claim 1, wherein said bone marrow stromal cells are rat bone marrow stromal cells.

11. The method of claim 1, wherein said NSCs are human NSCs.

12. The method of claim 1, further comprising administering to said recipient an immunosuppressive agent.

13. The method of claim 1, wherein said bone marrow stromal cells are administered to the recipient prior to said transplant.

14. The method of claim 1, wherein said bone marrow stromal stem cells are administered to the recipient concurrently with said transplant.

15. The method of claim 14, wherein said bone marrow stromal cells are administered as part of the transplant.

16. The method of claim 1, wherein said bone marrow stromal cells are administered to the recipient subsequent to the transplantation of the transplant.

17. The method of claim 1, wherein said bone marrow stromal cells are administered intravenously to the recipient.

18. The method of claim 1, wherein said effector cells are cells of a recipient of said donor transplant.

19. The method of claim 1, wherein said bone stromal cells are genetically modified.

20. A method for treating a transplant recipient, wherein said transplant is a neural stem cell (NSC), to reduce in said recipient an immune response of effector cells against an alloantigen to the effector cells, comprising:
transplanting to a transplant recipient NSCs with bone marrow stromal cells in an amount effective to reduce an immune response of effector cells against an alloantigen to the effector cells, whereby in the transplant recipient the effector cells have a reduced immune response against the alloantigen.

21. The method of claim 20 wherein said effector cells are T cells.
